(19) United States
(12) Patent Application Publication
(10) Pub. No.: US 2026/0311875 A1
(43) Pub. Date: Oct. 8, 2026

(54) SYSTEMS AND METHODS FOR DETECTION AND GRADING OF AGE-RELATED MACULAR DEGENERATION

(71) Applicant: University of Louisville Research Foundation, Inc., Louisville, KY (US)

(72) Inventors: Ayman El-Baz, Louisville, KY (US); Mohamed Elsharkawy, Louisville, KY (US); Ahmed Sharafeldeen, Louisville, KY (US); Fahmi Khalifa, Louisville, KY (US); Ahmed Soliman, Farragut, TN (US); Ahmed Elnakib, Erie, PA (US); Harpal Sandhu, Portland, OR (US)

(21) Appl. No.: 19/478,898

(22) PCT Filed: Apr. 26, 2024
(86) PCT No.: PCT/US2024/026556

§ 371 Date: Oct. 27, 2025

Related U.S. Application Data
(60) Provisional application No. 63/499,212, filed on Apr. 29, 2023.

(51) Int. Cl. G16H 50/20; G16H 30/40

(52) U.S. Cl. CPC G16H 50/20; G16H 30/40

(57) ABSTRACT

Computer-implemented system and methods for automated diagnosis of age-related macular degeneration apply machine learning techniques to optical coherence tomography B-scans to diagnose and grade age-related macular degeneration and other eye diseases.

### CROSS-REFERENCE TO RELATED APPLICATION

This Application claims the benefit of U.S. provisional patent application Ser. No. 63/499,212, filed Apr. 29, 2023, for SYSTEMS AND METHODS FOR DETECTION AND GRADING OF AGE-RELATED MACULAR DEGENERATION, incorporated herein by reference.

### FIELD OF THE INVENTION

Computer-implemented systems and methods for automated diagnosis of age-related macular degeneration apply machine learning techniques to optical coherence tomography B-scans to diagnose and grade age-related macular degeneration and other eye diseases.

### BACKGROUND OF THE INVENTION

Age-related macular degeneration (AMD) is one of the most common diseases affecting vision in the elderly, and it is projected that 6.3 million Americans will have AMD disease by 2030. In addition, the number of people in the world with AMD will grow up to 288 million by 2040. The macula is the center of the retina, which is responsible for high resolution central vision and can degenerate with aging and a combination of genetic and behavioral factors. Continuous follow-up with an ophthalmologist is recommended for those with signs of early AMD in order to detect progression to more advanced stages.

Typically, AMD has multiple different levels of severity, and in its advanced stages can take on different forms. The disease is broadly divided into two classes, dry and wet AMD. The earlier form is characterized by the presence of drusen of various sizes beneath the retina or retinal pigment epithelium (RPE). While there are several different classification schemes for AMD, the number and size of drusen contribute to distinctions between early versus intermediate dry AMD. Based on early studies and later age-related eye disease clinical trials, patients with intermediate dry AMD benefit from dietary and vitamin supplementation to slow the progression of AMD towards its two advanced forms, geographic atrophy (GA) and wet AMD. GA is an advanced, atrophic form of dry AMD, where the cells in the outer retina and RPE die, causing progressive vision loss. Wet AMD is characterized by ingrowth of pathologic, new blood vessels beneath or into the retina, called choroidal neovascularization (CNV). Retinal angiomatous proliferation is another form of wet AMD called type 3 neovascularization. These vessels are incompetent and thus leak fluid beneath the retina (subretinal fluid, or SRF) and/or into the retina (intraretinal fluid, or IRF). IRF and SRF are forms of exudation. Here, active wet AMD is defined by the presence of IRF, SRF or both, while inactive wet AMD has no fluid present.

Clinically, optical coherence tomography (OCT) has proven invaluable for the diagnosis and management of AMD as it provides detailed, in vivo images of the human macula at resolutions of 5-7 microns. Additionally, it offers numerous advantages over clinical examination of color fundus photographs (CFP), such as the ability to identify subtle amounts of IRF or SRF and the ability to measure the central macular thickness. The advantage of CFP over OCT is the identification of shallow subretinal hemorrhage, which can be difficult with OCT.

In order to predict AMD progression using OCT, multiple prior computer-aided diagnosis (CAD) systems for grading AMD have been developed. Most recent CAD systems have been using deep learning technique, which is the state-of-the-art technology that achieves promising results. The common drawbacks of these methods are that (i) they extract features from CNN layers that may be difficult to interpret clinically and do not depend on clinical descriptors of AMD and (ii) they do not differentiate between AMD grades which carry different prognoses, e.g., CNV can itself be active or inactive; in addition, as for drusen, it can be in an early, intermediate, or advanced form (GA).

Other automated systems have been introduced that utilize machine learning (ML) algorithms to predict different grades of AMD. In contrast, other automated systems used CFP to differentiate between the normal and different AMD grades. However, these systems do not depend on clinical findings that are important for ophthalmologists to identify different stages of disease and they do not identify inactive wet AMD. Another system includes unsupervised learning methods but it was found that new, unknown images may produce different outcomes than expected because in unsupervised learning there are no notions of the output during the training process.

Despite the fact that there has been significant progress in AMD diagnosis using OCT, the existing studies have some limitations: (i) there is no system that differentiates between normal images, all fives grades of AMD, and other eye diseases, (ii) most CAD systems have tended to offer cruder outputs, such as the presence of exudation versus no exudation, or positive diagnosis of AMD versus no AMD, (iii) the presented deep CNN models deal with the images as black boxes with no clinically meaningful descriptive features, and (iv) CFP cannot detect some of the most important signs of wet AMD activity, namely, SRF and IRF, in the vast majority of cases.

### SUMMARY

The inventors present a computer-aided diagnostic (CAD) system and method for diagnosing and grading patients with AMD based on OCT images with a level of granularity that mirrors the important clinical distinctions that retinal specialists themselves make. The disclosed CAD system and method to differentiate between normal retina, the grades of AMD, and non-AMD diseases includes five steps as summarized in FIG. 1: (i) OCT image preprocessing, (ii) abnormality detection, and (iii-v) up to three sequential stages of grading. Certain grades of AMD and non-AMD diseases are diagnosed in the first grading stage, and if not, the system proceeds to the second grading stage to diagnose other grades of AMD, non-AMD diseases or normal retina. In the event the OCT image is classified as indicative or early or intermediate AMD in the second stage, the system proceeds to the third grading stage to differentiate between early and intermediate AMD.

The system distinguishes medical images indicative of the presence of AMD from images indicative of normal retina, and further grades images indicative of AMD as early dry AMD, intermediate dry AMD, GA, and wet AMD. Wet AMD is further classified as either inactive wet AMD or active wet AMD, a critical distinction that informs a patient's treatment regimen. There are multiple causes of maculopathy that present with OCT abnormalities apart from AMD. Accordingly, the disclosed CAD system is also configured to identify medical images indicative of abnormal retina which are not indicative of AMD. Exemplary non-AMD disease states which present OCT abnormalities include diabetic retinopathy (DR), Stargardt disease, epiretinal membranes (ERM), and macular hole (MH).

To improve upon and avoid the limitations of existing OCT-based methods of AMD diagnosis, the inventors disclose an automated CAD approach that differentiates between a healthy macula and the five clinical classes of AMD (i.e., early, intermediate, GA, active wet, and inactive wet AMD), and non-AMD eye diseases.

The disclosed system detects from medical images medical landmarks of AMD, which are clinically meaningful and readily identifiable by experts. These medical landmarks show macular abnormalities on OCT cross-sectional images referred to as B-scans, e.g., detecting subretinal or sub-RPE tissue, retinal fluid (IRF and SRF), detecting choroidal hypertransmission (CH), detecting discontinuities/loss of OCT layers (merged layers), and detecting and calculating features-based segmented OCT layers (drusen detection using the estimated 2D curvature, thickness, first-order reflectivity, local and global high-order reflectivity based on gray-level co-occurrence matrix (GLCM) and Markov-Gibbs random field (MGRF), respectively).

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 depicts a schematic summary of the CAD system for AMD diagnosis using OCT images. This comprehensive procedure to differentiate between normal retina, the grades of AMD, and non-AMD diseases includes OCT image preprocessing and three sequential stages of grading.

FIG. 2 depicts an illustrative encoder-decoder architecture of DeepLabv3+ with ResNet50 CNN as a backbone. First, a ResNet50 backbone using Atrous convolutions processes the input OCT images, resulting in feature maps. Additional processing is then applied to the ReNet50 output (feature maps) by Atrous spatial pyramid pooling (ASPP). The low-level features extracted from the ResNet50 network are concatenated with the output of the encoder. Lastly, the decoder estimates the predicted semantic labels.

FIG. 4 depicts examples of the first grading system based on the retinal abnormalities detection. The first and second row show examples of non-AMD diseases (MH and ERM, respectively). The third row shows an example of active wet AMD. The fourth row shows an example of inactive wet AMD. The fifth and sixth rows show examples of GA and non-AMD disease (Stargardt). After detection of MRL and CH for those two diseases, KNN classifier-based features are used to distinguish between GA and non-AMD disease (Stargardt). MRL: merged layer and CH: choroidal hypertransmission.

FIG. 5 depicts an illustration of the classifier for second-stage grading. First, three features are extracted from each retinal layer. Then, the extracted features are fused and used to classify each layer. Then, a majority voting for all layer classifications is applied to generate the final diagnosis for the second stage.

FIG. 6A illustrates use of the Menger curvature formula using circles to estimate the curvature of a retinal layer at points m and n, with relatively high values of curvature indicating the presence of drusen.

FIG. 6B illustrates estimation of thickness of a retinal layer as used in the second grading stage. The four panels depict, left-to-right, OCT images of retina displaying a non-AMD disease state (diabetic retinopathy), intermediate AMD, early AMD and normal state.

FIG. 6C illustrates estimation of Gibbs energy at point from its fourth-order neighborhood pixels as used in the third grading stage.

FIG. 6D illustrates estimated normalized GLCM based on the number of times each pixel value at a given point p0 appears with its eight neighbor pixels at distance r as used in the third grading stage.

FIG. 7 depicts validation loss vs. training loss during training of the first grading stage with respect to tissue detection (top left), fluid detection (top right), choroidal hypertransmission (CH) (bottom left) and merged layer detection (bottom right).

### DETAILED DESCRIPTION OF THE PREFERRED EMBODIMENTS

For the purposes of promoting an understanding of the principles of the invention, reference will now be made to selected embodiments illustrated in the drawings and specific language will be used to describe the same. It will nevertheless be understood that no limitation of the scope of the invention is thereby intended; any alterations and further modifications of the described or illustrated embodiments, and any further applications of the principles of the invention as illustrated herein are contemplated as would normally occur to one skilled in the art to which the invention relates. At least one embodiment of the invention is shown in great detail, although it will be apparent to those skilled in the relevant art that some features or some combinations of features may not be shown for the sake of clarity.

Any reference to “invention” within this document is a reference to an embodiment of a family of inventions, with no single embodiment including features that are necessarily included in all embodiments, unless otherwise stated. Furthermore, although there may be references to “advantages” provided by some embodiments of the present invention, other embodiments may not include those same advantages, or may include different advantages. Any advantages described herein are not to be construed as limiting to any of the claims.

Specific quantities (spatial dimensions, dimensionless parameters, etc.) may be used explicitly or implicitly herein, such specific quantities are presented as examples only and are approximate values unless otherwise indicated. Discussions pertaining to specific compositions of matter, if present, are presented as examples only and do not limit the applicability of other compositions of matter, especially other compositions of matter with similar properties, unless otherwise indicated. Unless stated otherwise, explicit approximate quantities (e.g., about 1; approximately 20) refer to a range of ±10% of the recited quantities (e.g., “about 1” refers to 0.9 to 1.1; “approximately 20” refers to the range of 18 to 22).

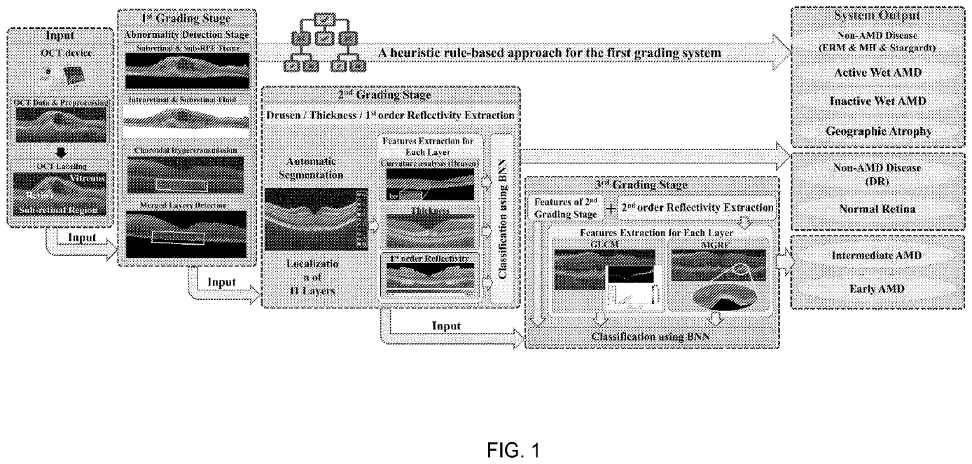

Referring now to FIG. 1, the proposed CAD system to differentiate between normal retina, the grades of AMD, and non-AMD diseases broadly includes five steps: (i) OCT image preprocessing, (ii) abnormality detection, (iii-v) three sequential stages of grading. As explained in further detail, the first grading stage categorizes the OCT image as indicative of non-AMD disease, active wet AMD, inactive wet AMD.

### Preprocessing

Before actual analysis, the system includes an image normalization and a region of interest (ROI) selection. To reduce the variability in the signal-to-noise ratio between the OCT images, all images are normalized to be in the range 0-1. The ROI corresponding to the retina region is delineated using the inventors' multiresolution edge detector described in A. El Tanboly et al., “A novel automatic segmentation of healthy and diseased retinal layers from OCT scans,” in Proc. IEEE Int. Conf. Image Process., 2016, pp. 116-120 (“El Tanboly et al.”). In the following sections, abnormality detection as well as the grading stages are fully described.

### Abnormality Detection

The analysis pipeline begins by identifying a set of diagnostic features that are sufficient for the first grading stage. These grading medical landmarks are (i) subretinal and sub-RPE tissue, (ii) fluids (IRF and SRF), (iii) CH, (iv) detecting discontinuities/loss of OCT layers, (i.e., merged layers).

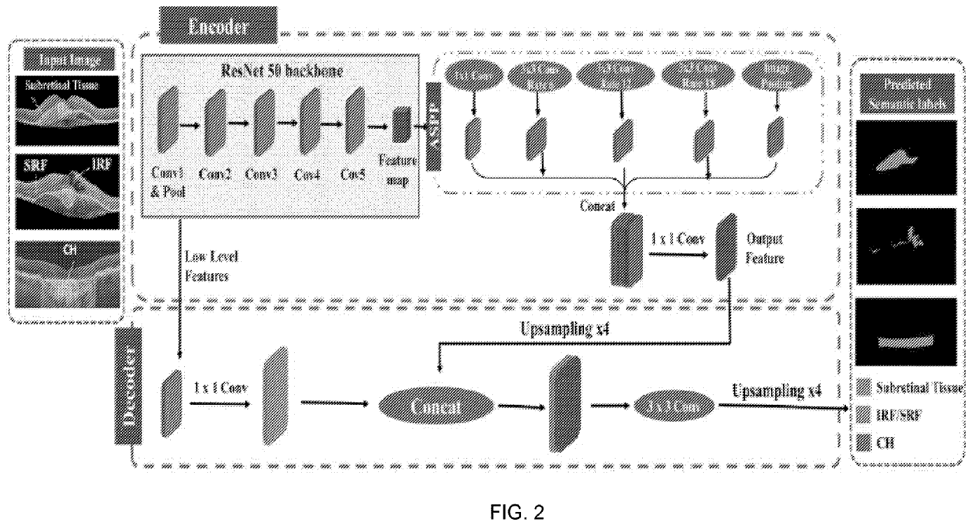

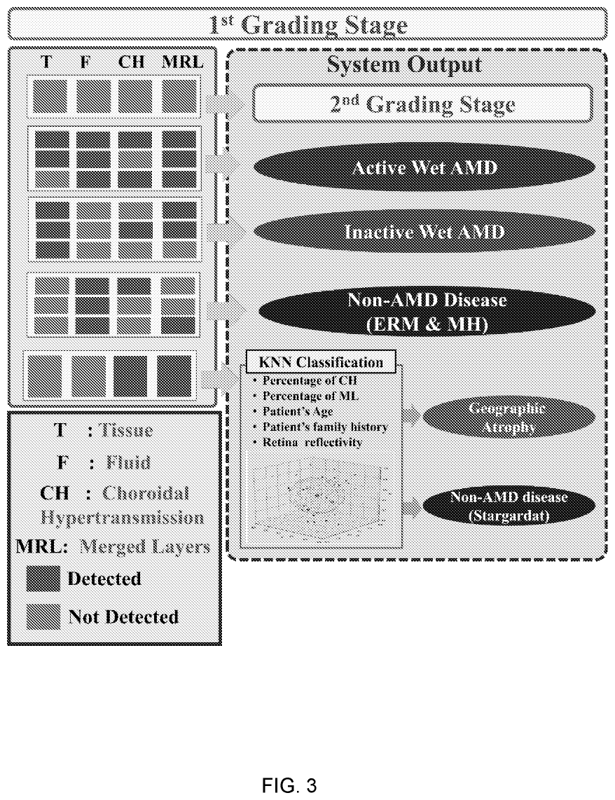

Fluid, Tissue and Choroidal Hypertransmission Detection: In an embodiment of the system, a semantic segmentation network, such as, for example, the DeepLabV3+ network, is utilized to detect image anomalies regions corresponding to subretinal tissue, sub-RPE tissue, IRF, SRF, and CH. DeepLabV3+ is designed for semantic segmentation, namely, classifying each pixel in an image into a class, and is composed of an encoder-decoder network as illustrated in FIG. 2. First, the input OCT image is processed by a CNN, such as, for example, ResNet50, which works as a backbone for DeepLabV3+. The ResNet50 backbone is implemented using a multiscale processing module using Atrous spatial pyramid pooling (ASPP) is applied to the ResNet50 output. Three Atrous convolutions are used with large Atrous rates (6, 12, and 18). The Atrous controlled the impact of its area of view using the control rate parameter, (r):

$${m}_{out} ( p ) = {\sum }_{c = 1}^{c} {n}_{inp} [ p + rc ] \cdot {w}_{f} [ c ] \qquad ( 1 )$$

where w_{f}[c] is a filter with length C, nᵢₙₚ[i] is the input pixel p, and mₒᵤₜ(p) is the output pixel. Then, the output from ASPP results in extracting dense feature maps (the output from the encoder stage). In the decoder stage, the output encoded feature maps are upsampled by a factor of four and concatenated with the low-level features obtained from the ResNet50 backbone. To ensure that the dense features extracted from the encoder are not overshadowed, it is preferable to reduce the number of channels in the low-level features before the concatenation. The low-level features typically contain a large number of channels such as 256 or 512, which may dominate the significance of dense features extracted from the encoder. For this reason, a 1×1 convolution is applied to the low-level features. Finally, the decoder works on estimating the predicted semantic labels by applying 3×3 convolutions to refine the features, then upsampling these features by a factor of four to produce an output segmentation equal in size to the input. When tuning the DeepLabV3+ network, class-weight balancing is used to reduce bias, since the area of abnormal regions is generally much smaller than the area of the retina region as a whole. This class imbalance is addressed using the weighted cross-entropy function.

Merged Layer Detection: The aim of this step is to identify regions of retinal or RPE atrophy, where one or more retinal layers typically seen in an OCT image are absent, i.e., the layers are collapsed or merged with adjacent layers. To accomplish this task, a CNN network analyzes OCT images to distinguish merged retinal layers from unmerged retinal layers. The CNN network learns features of merged or unmerged layers through convolutional layers, then classifies regions based on these features in fully connected layers. The changes/merging in the retinal layers are very small and any downsampling in the OCT images may lead to missing crucial information. In some embodiments, the disclosed CNN model includes ten different processing blocks. Each block starts with a convolution layer, a batch normalization layer to speed up the training process, a rectified linear unit (ReLU) activation layer, to mitigate the vanishing gradient problem, and finally, an average pooling layer is employed at the end of each block. At the end, a flattening operation is applied on the feature map, resulting from the final average pooling layer, to have a one-dimensional feature map. The fully connected layer consists of two neurons for the two classes (merged or non-merged). Also, the final classification layer uses the softmax activation function.

### First Grading Stage

In this stage, the system classifies the OCT image as indicative of active wet AMD, inactive wet AMD, GA, non-AMD diseases such as Stargardt, ERM, and MH. If the OCT is image is not classified as indicative of active wet AMD, inactive wet AMD, GA, or a non-AMD disease, the system proceeds to the second grading stage. The system classifies the OCT image in this first grading stage based on the detection or absence of the four diagnostic features (e.g., (i) subretinal and sub-RPE tissue (collectively, “Tissue” FIG. 3), (ii) IRF and/or SRF (collectively, “Fluid” in FIG. 3), (iii) CH, and (iv) merged layers, abbreviated as “MRL” in FIG. 3). The classification algorithm employed in the first grading system follows a heuristic rule-based approach as depicted in FIG. 3. As indicated in the figure, the system classifies a retinal OCT image as indicative of active wet AMD upon detection of Tissue, Fluid, CH and MRL, or detection of Tissue, Fluid and MRL but not CH, or detection of Fluid, CH and MRL, but not Tissue. The system classifies a retinal OCT image as indicative of inactive wet AMD upon detection of Tissue and MRL but not Fluid and CH, or detection of Tissue, CH and MRL, but not Fluid, or detection of Tissue but not Fluid, CH and MRL. The system also identifies severe non-AMD diseases (MH and ERM) upon detection of Fluid and CH but not Tissue and MRL, or detection of Fluid but not Tissue, CH and MRL, or detection of Fluid and MRL but not Tissue and CH. For GA and non-AMD disease (Stargardt), their characteristics in OCT images are very close to each other. Stargardt disease is an inherited macular degeneration, usually presents in the first three decades of age, and causes progressive atrophy in the macula, similar to GA secondary to AMD. In both, the system detects CH and MRL but does not detect Fluid and Tissue. To make a clinical diagnosis of Stargardt disease, retina specialists rely on the age of diagnosis as a key variable. Here, the CAD system also includes as inputs the age of individual whose retina is depicted in the OCT image and whether the individual has a family history of Stargardt disease. To differentiate between GA and Stargardt, the CAD system uses a K-nearest neighbor (KNN) classification in the feature space comprising: the percentage of merged layers, percentage of CH, retina reflectivity, normalized age, and family history. In instances where the CAD system detects the characteristics of any non-AMD disease, it generates a diagnosis of non-AMD disease. In other embodiments, the CAD system may identify the specific non-AMD disease detected. In the event the CAD system detects none of Tissue, Fluid, CH and MRL, it proceeds to the second grading stage.

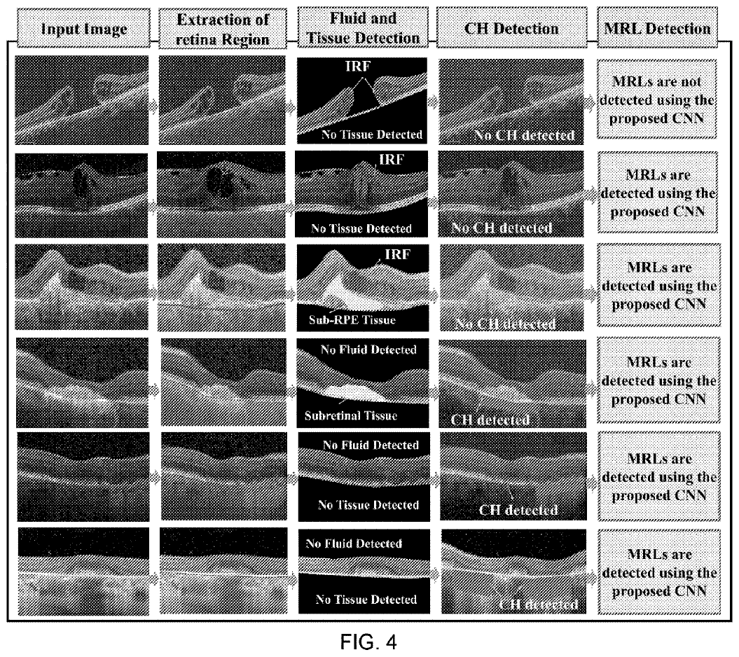

FIG. 4 depicts, in each row, left to right, (1) an exemplary OCT image of a subject retina, (2) the image labeled and the retina detected, (3) the image with subretinal tissue, sub-RPE tissue, IRF and SRF labeled, if detected, (4) the image with CH labeled, if detected, and (5) an indicator of whether merged retinal layers (MRL) are detected using a CNN.

### Second Grading Stage

OCT images not classified in the first grading stage proceed to the second grading stage, which aims to distinguish between normal, nonadvanced (early and intermediate) AMD, and non-AMD disease (e.g., DR) based on the extracted features from the segmented OCT layers. Thus, OCT layer segmentation is required. To achieve this, the system uses the inventors' previously developed atlas-based approach as described in A. El Tanboly et al., “A novel automatic segmentation of healthy and diseased retinal layers from oct scans,” IEEE International Conference on Image Processing (ICIP), pp. 116-120, 2016, to automatically segment eleven retinal OCT layers.

For each segmented layer, the system detects drusen through the analysis of the estimated 2D curvature. Furthermore, the system estimates the thickness and reflectivity features to be able to differentiate between the three classes in the second grading stage, which is described next.

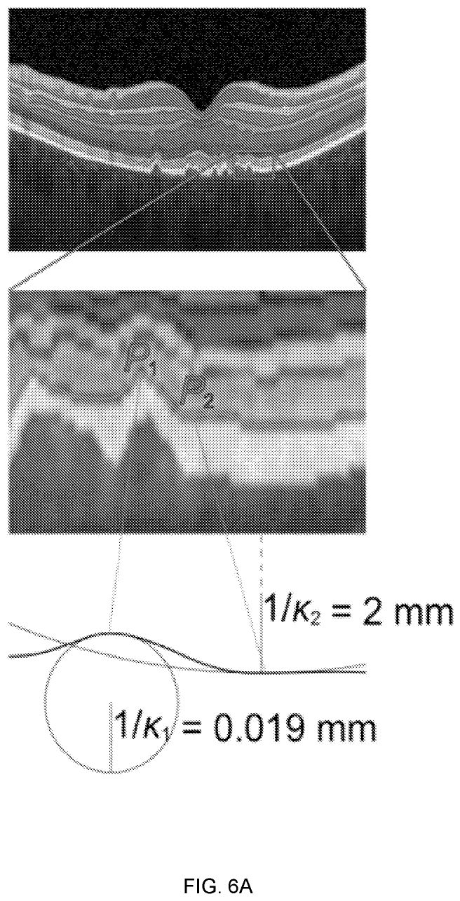

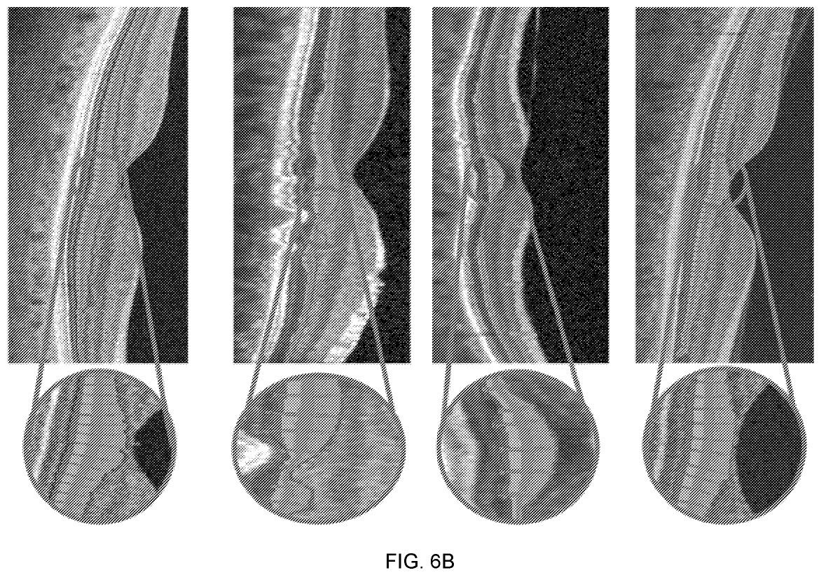

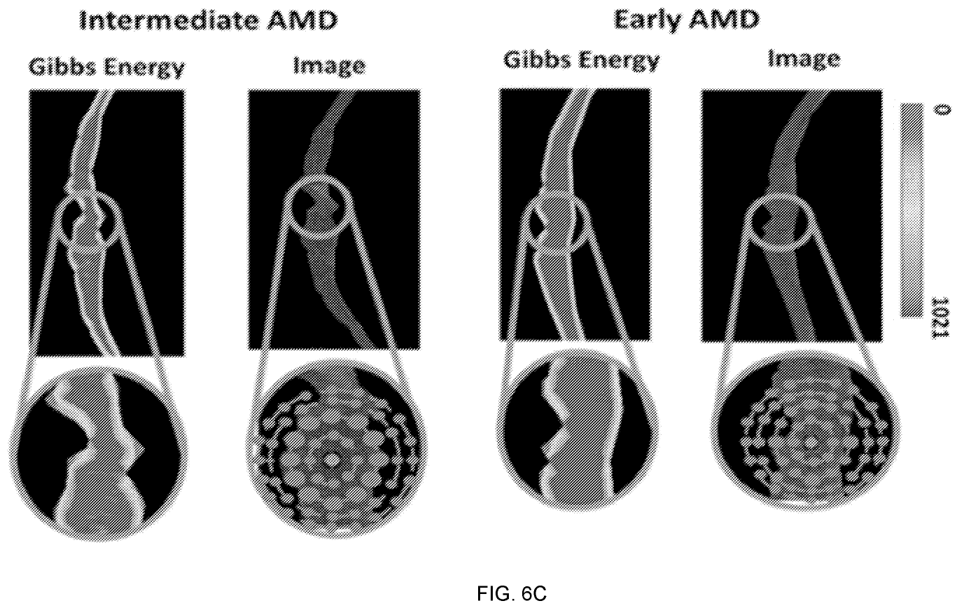

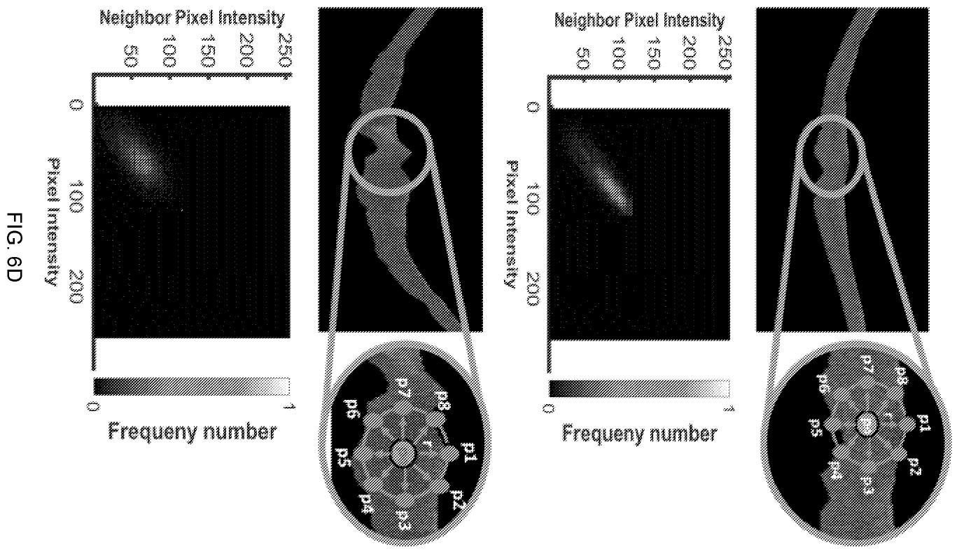

Drusen Detection: Drusen detection searches for bending or warping of the retinal layers that result from drusenoid deposits. This assumes the retinal layer structure is otherwise intact, as in normal retina, or has abnormal curvature from the presence of drusen, as in non-advanced AMD cases (early and intermediate AMD), but there are no merged retinal layers, as OCT images with merged layers were classified in the first grading stage. A local measure of bending or curvature, K is calculated for each layer using a three points circle to estimate absolute curvature. The basic steps for calculating the estimated curvature at a given layer are presented in Algorithm 1. Curvature is estimated locally in the neighborhood of each pixel location on the midline of the retinal layer. This produces a large and variable number of curvature measurements per layer. Due to the varying sizes of each layer, the curvature feature is measured at distinct bins of the segmented B-scan and the average value of K values at each bin is computed. An example of an OCT image and determination of K values is depicted in FIG. 6A. For better representation and generalizability for these values, the cumulative distribution function (CDF) is employed. In some embodiments, only a representative sample of the statistical measures of the CF, such as, for example, nine percentiles of the thickness values, are taken to create the thickness vector descriptor. Pseudo-code of the curvature calculation is provided in Algorithm 1.

Algorithm 1: Pseudo-code of the curvature calculation

Input: P₁, P₂, and P₃ are three given points at the mid-line boundary of a
given layer.
Output: κ is the estimated curvature.
Procedure Curvature(P₁, P₂, P₃)
If (P₁, P₂, P₃) = collinear then
κ = 0
else
$radius = \frac{‖ P 1 - P 2 ‖ ‖ P 2 - P 3 ‖ ‖ P 3 - P 1 ‖}{4 S ( P 1 , P 2 , P 3 )}$
S(P₁, P₂, P₃) is the area of a triangle P₁, P₂, P₃
κ = radius⁻¹

Adjusting the spacing (i.e., the arc length on the mid-line) between the three points allows for estimating curvature at coarser scales.

Thickness: Retinal layer thickness is calculated by measuring the distance between the inner and outer boundaries of that layer. Considering that the boundaries are curved, an unambiguous notion of distance from one to the other is defined in terms of the solution of Laplace equation. Considering the outer and inner boundaries as equipotential surfaces, each point on one boundary is joined to a corresponding point on the other by a unique “field line”. These field lines do not cross, and any point within the layer lies on one and only one field line. Finally, the Laplace equation is solved within the retina layer using the iterative Jacobi approach:

$${γ}^{i + 1} ( x , y ) = \frac{1}{4} \{ {γ}^{i} ( x + Δ x , y ) + {γ}^{i} ( x - Δ x , y ) + {γ}^{i} ( x , y + Δ y ) + {γ}^{i} ( x , y - Δ y ) \} \qquad ( 2 )$$

where yⁱ(x,y) is the potential at interior point (x,y) on iteration i, and Δx (and respectively, Δy) is the pixel spacing in the x (and respectively y) direction. The potential is initialized to y⁰(x,y)=0, and fixed at y=−1 on the inner boundary and yⁱ=1 on the outer boundary for all i. Iterating until there is no appreciable change in y, the field lines l satisfy l=∇y(l). The use of a geometric approach instead of localizing the point pairs solves the problem of variations of signal intensity, which appears in retinal layers. Finally, a CDF vector of thickness values throughout each layer is constructed. In some embodiments, only a representative sample of the thickness values, such as, for example, nine percentiles of the thickness values, are taken to create the thickness vector descriptor. FIG. 6B depicts exemplary OCT images of retina displaying, left-to-right, a non-AMD disease state (diabetic retinopathy), intermediate AMD, early AMD and normal state. The expanded areas of each image show calculated thickness values.

Reflectivity: Reflectivity of a retinal layer in an OCT image is calculated as the mean value of pixels within that layer. Prior to calculation, the image is normalized in order that scans of different retinas have the same dynamic range, and the pixel intensities are comparable. Normalization is performed according to Eq. 3.

$${I}_{Nimg} = 200 \times \frac{{I}_{img} - {R}_{Vitreous}}{{R}_{RPE} - {R}_{Vitreous}} + 27.5 \qquad ( 3 )$$

where I_{Nimg} is the normalized image, I_{img} is the original image, Rᵥᵢₜᵣₑₒᵤₛ and R_{RPE} are the mean unnormalized intensities of image regions labeled vitreous and RPE, respectively. Resultant normalized intensities are rounded and converted to 8-bit integers, with values below 0 or greater than 255 being clipped. The reflectivity feature of each layer is derived from the CDF of its normalized pixel values. In some embodiments, it is that portion of the CDF between the 10th and 90th percentile of normalized gray levels, so that extreme values are discarded.

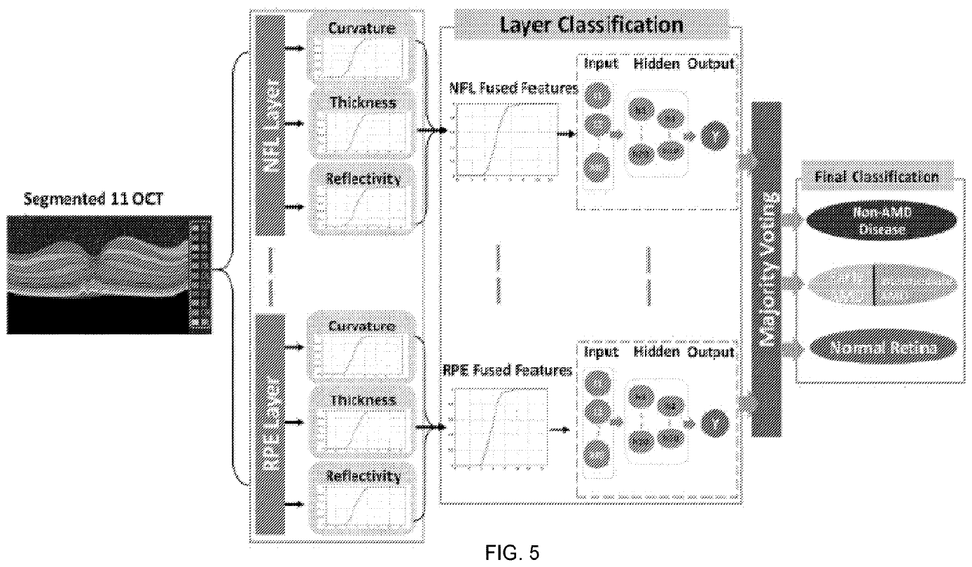

Finally, the three constructed CDFs from these features (i.e., curvature, thickness, and reflectivity) are fused to generate a final vector descriptor for each OCT layer. Then, this final vector is fed to a backpropagation neural network (BNN) classifier that determines whether the layer is distorted by the presence of drusen or distorted by the calculated thickness and reflectivity to generate a diagnosis of (i) normal, (ii) early or intermediate AMD, or (iii) non-AMD disease for each layer. The final diagnosis for this section stage is based on a majority voting of the BNN-generated diagnoses generated for all eleven retinal layers. The aforementioned steps of the second grading stage are illustrated in FIG. 5. Note that in certain embodiments, the final diagnosis of the second grading stage is generated based on majority voting with a greater weight applied to the diagnoses generated for the ellipsoid zone (EZ), outer photoreceptor (OPR) segments, and RPE retinal layers. In further embodiments, the final diagnosis of the second grading stage is generated based on majority voting of only the diagnoses generated for the EZ, OPR segments, and RPE retinal layers.

### Third Grading Stage

In this stage, the system differentiates between the two less severe grades of AMD (early and intermediate). The determination is made by evaluation of the same features extracted from the segmented retinal layers as in the second grading stage (curvature, thickness and reflectivity) in addition to 2^{nd}-order reflectivity features derived from MGRF and GLCM models. Both of these two models describe the texture homogeneity to provide an additional distinction between these two grades as they are very close to each other. In the following subsections, the details of these two models are illustrated.

2nd-order Reflectivity based on MGRF: To model the correlation between image gray levels at a second-order level, each OCT image is considered as a specific instance of a MGRF. The MGRF is characterized by a network of relationships among pixels that belong to a fully connected neighborhood, or clique. Specifically, an MGRF that has a translation-invariant system with four neighboring pixels is implemented.

The notation can be defined as (1) g:R→Q is a grayscale image on the discrete domain R⊂ [symbol] × [symbol] with pixel values in Q={0, . . . , Q−1}, (2) N={(ξᵢ,ηᵢ), i=1, . . . , η} is a set of (x,y)-offsets specifying the pairwise neighborhood system, (3) C is the graph of pixel interactions on R; the neighborhood system for pixel (x,y)∈R is the set of pairs C(x,y)={{(x,y),(x+ξ,y+η)}, (ξ,η)∈N}, and (4) Vᵢ:Q×Q→ [symbol] is the Gibbs potential associated with neighborhood (ξᵢ,ηᵢ).

With these preliminaries, the second order MGRF on R is specified by its Gibbs probability distribution function:

$$P ( q ) = \frac{1}{Z} exp ( \vert R \vert {\sum }_{i = 1}^{n} {\sum }_{{q}^{′} = 0}^{Q - 1} {V}_{i} ( q , {q}^{′} ) {F}_{i} ( q , {q}^{′} ) ) \qquad ( 4 )$$

The aforementioned notation involves Z, which represents the partition function, |R|, which denotes the size of R, and Fᵢ, which is the scaled gray level co-occurrence matrix or the empirical bivariate probability distribution for the neighborhood family i. The estimation of potentials Vᵢ in equation 4 is crucial in determine the MGRF model. The analytical maximum likelihood estimator for the 2^{nd}-order Gibbs potentials is employed to achieve this estimation,

$${V}_{i} ( q , s ) = {ρ}_{i} \cdot ( {F}_{i} ( q , s ) - {f}_{i} ( q ) {f}_{i} ( s ) ) , \qquad ( 5 )$$

The calculation of Gibbs potentials is done on a per-layer basis, where

$${f}_{i} ( q ) = \frac{1}{Q} {\sum }_{{q}^{′} = 0}^{Q - 1} {F}_{i} ( q , {q}^{′} )$$

denotes the gray level marginal distribution. Hence, for each retinal layer I present in the OCT images, a vector of Gibbs energies is computed to describe the texture of that specific layer,

$${E}_{l , i} = {\sum }_{q = 0}^{Q - 1} {\sum }_{{q}^{′} = 0}^{Q - 1} {F}_{l , i} ( q , {q}^{′} ) ( {F}_{l , i} ( q , {q}^{′} ) - {f}_{l , i} ( q ) {f}_{l , i} ( {q}^{′} ) ) \qquad ( 6 )$$

A visualization of the estimated Gibbs energy values can demonstrate the significant contrast between the values on the curves as shown in FIG. 6C. The Gibbs energy values of a layer are summarized using a vector descriptor. To construct this descriptor, the CDF of Gibbs energy values for the layer is computed, and then a representative sample of the statistical measures of the CF, such as, for example, nine percentiles of the Gibbs energy values, are selected from the CDF and used to create the vector descriptor, which captures the essential characteristics of the Gibbs energy values for the layer.

2^{nd}-order Reflectivity based on GLCM: A second order reflectivity based on the GLCM model is also used in the system to represent the connectivity of 8-neighbors for each pixel in the OCT image for each OCT layer. The values of GLCM represent how many times the pixels' values appear together. Then, a GLCM is normalized to extract statistical features in order to represent the relationships between each pixel and its 8-neighbors. The extracted features from GLCM are contrast, correlation, energy, and homogeneity. Contrast measures the variation occurred in intensity levels between each pixel and its 8-neighbors, while correlation represents how each pixel correlate with its 8-neighbors. Energy is used to represent the percentage of variation of intensity values. For example, if the energy value is equal to 1, then the image will be fully homogeneous. Last, the homogeneity feature shows how GLCM elements appear concentrated around its diagonal, as shown in FIG. 6D. GLCM matrices obtained from OCT images of retina experiencing early AMD and intermediate AMD show different ranges of values.

Finally, the constructed CDFs for curvature, thickness and reflectivity created in the second grading stage are combined with the 2^{nd} order reflectivity based on MGRF and 2^{nd} order reflective based on GLCM created in the third grading stage are fused to create the final vector descriptor for each OCT layer in this third grading stage. Then, a layerwise classification is performed to generate a diagnosis for each layer. Lastly, a majority voting is applied to the eleven layers' diagnoses to generate the final diagnosis and classify the OCT image as indicative of either early AMD or intermediate AMD.

The basic steps of the first, second and third grading systems are summarized in Algorithm 2.

Algorithm 2: Basic Steps of the grading system
First grading system:

| | |
|---|---|
| 1. | Detect IRF and SRF using DeepLabv3+. |
| 2. | Detect Sub-RPE tissue and subretinal tissue using DeepLabv3+. |
| 3. | Detect CH using DeepLabv3+. |
| 4. | Detect merged layers using the proposed CNN model. |
| 5. | Use the results obtained from Steps 1 to 4 to classify the OCT image as |
| | active wet AMD, inactive wet AMD, GA, and non-AMD diseases (e.g., ERM, |
| | MH, Stargardt). |

Second grading stage:

| | |
|---|---|
| 1. | Segment the eleven OCT layers for OCTs without any abnormalities detected |
| | in the first stage using the technique described in El Tanboly et al. |
| 2. | Detect drusen using the estimated 2D curvature for the segmented layers. |
| 3. | Estimate thickness and reflectivity for the segmented layers, construct the |
| | CDFs for the extracted features from the retina layers and obtain the 10th |
| | through 90th percentiles. |
| 4. | Fuse the constructed CDFs for each layer and apply BNN layer-wise |
| | classification output for each layer. |
| 5. | Apply majority voting to get the final diagnosis of the OCT (e.g., normal or |
| | non-severe AMD (early and intermediate) or non-AMD disease (e.g., DR). |

Third grading stage:

| | |
|---|---|
| 1. | Segment OCT layers using the technique described in El Tanboly et al. |
| 2. | Estimate second-order reflectivity features using MGRF and GLCM in addition |
| | to the three features discussed in the second grading stage. |
| 3. | Feed the estimated CDFs extracted from each layer to BNN to get layer |
| | diagnosis. |
| 4. | Apply majority voting to get the final diagnosis of the OCT (early AMD or |
| | intermediate AMD). |

### Experimental Results

The disclosed CAD system was tested on OCT images collected in a retrospective study for patients with clinical diagnosis of AMD and non-AMD diseases at two different referral centers in the U.S., the University of Louisville and Legacy Devers Eye Institute (LDEI). OCT imaging was performed using Zeiss Cirrus OCT models 4000 and 5000 at the University of Louisville and a Heidelberg device at LDEI.

The resolution of the macular B-scans is either 1024×1024 or 1024×512 pixels, spanning an area of 2 mm deep and 6 mm from side to side. Pixel spacing is 1.955 μm along the anterior-posterior axis and either 5.865 μm or 11.74 μm along the naso-temporal axis. For a given eye, a single OCT B-scan was selected for analysis. Specifically, the single horizontal slice passing directly through the fovea was used. AMD grading was performed by two retinal experts in a two-phase process in which blind grading by each expert was first conducted followed by an unblinded phase where both experts agreed on a final grade for cases in cases where they originally disagreed. The dataset consisted of 1285 OCT B-scans from 167 patients, where multiple images collected from the same patient at different time points during the disease progression were included. The dataset was divided into 740 images for the purpose of training and validation on the three grading stages, and 545 images to test the system. The number of images for normal, early AMD, intermediate AMD, GA, inactive wet, active wet, and non-AMD diseases were 157, 122, 168, 122, 211, 220, and 285, respectively.

Fine-tuning of DeepLabv3+ to detect retinal abnormalities was done using optimization parameters. To determine these optimization parameters for the training, the grid search approach was used to obtain the best optimizer to optimize the loss function. The search space for the optimizer was adaptive moment estimation (Adam) and stochastic gradient descent with momentum (SGDM). The momentum value was set to 0.9, and the initial learning rate was set to 0.01 and decreased by a factor of 10 for every 5 iterations.

Further, to fine-tune the proposed merged layers' detection CNN, the same searching space for DeepLabV3+ tuning was used to choose the best optimizer. The momentum value is set to 0.9, and the initial learning rate is set to 0.001 and decreased by a factor of 10 for every 5 iterations. For the second grading stage, the BNN was trained using the Levenberg-Marquardt algorithm. Hyperparameter tuning to optimize network architecture included varying the hidden layer number (1 or 2) and sizes (5-20 neurons, inclusive). The preferred configuration for the BNN following tuning was two hidden layers with 15 and 7 neurons, respectively. For the third grading stage, the same hyperparameters were tuned for BNN using the Levenberg-Marquardt algorithm. Also, for the hidden layers, the search space was 1 and 2. But the search space for the number of neurons in each hidden layer was 5-35 as the number of features increased. The resulting BNN for the third grading stage had two hidden layers of size 20 and 10 neurons, respectively.

The training setup for each grading stage in the disclosed CAD system includes using a portion, such as, for example, ten percent, of the training set as a validation set for each of the three grading stages. For the detection of subretinal and sub-RPE tissue, 520° C. T B-scans were used to train the DeepLabv3+(260 images have sub-RPE and/or subretinal tissue as found in active and inactive wet AMD and 260 images are from other grades with no tissue abnormalities). For the detection of IRF and SRF, 260 images are used to train the DeepLabv3+(130 images including IRF and/or SRF as found in active wet AMD and 130 images from other grades without IRF or SRF). For the detection of CH, 260 images are used to train the DeepLabv3+(130 images have CH as found in GA and 130 images from other grades without CH). For the detection of merged layers, 480 images were used for training (240 images including merged layers as found in GA, inactive wet AMD and active wet AMD, and 240 images including non-merged layers as found in early AMD, intermediate AMD and normal retina).

To overcome the issue of having a limited number of training samples, a data augmentation strategy was adopted during the training process to avoid the problems of bias and overfitting. The augmentation strategy included rotations of up to 30° and changes in horizontal or vertical scale by up to 10% to create additional OCT images and augment the training dataset.

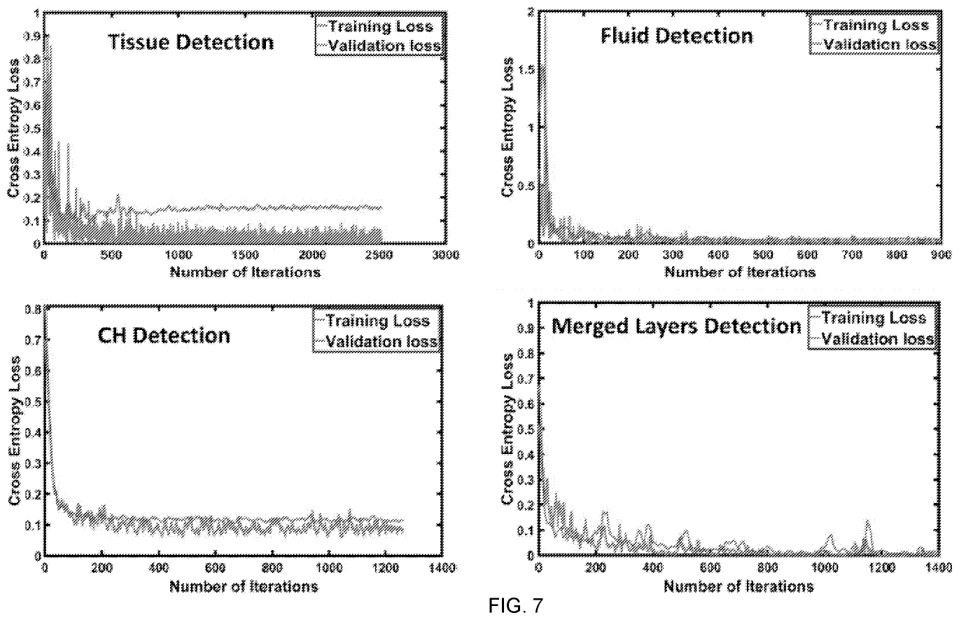

In order to get the best training models in the first grading stage for DeepLabV3+ to detect the macula abnormalities, the maximum number of epochs was set to 25 and the validation loss was monitored to determine the minimum value. Validation loss in detection of tissue abnormalities (i.e., subretinal and sub-RPE tissue), fluid (i.e., IRF and/or SRF), CH and merged layers are depicted in FIG. 7. The trained models for DeepLabV3+ were then saved to detect the retinal abnormalities. After the training process, the learned DeepLabv3+ models for fluid, tissue, and CH are used to detect anomalies in the test data. Abnormalities in the retina were successfully detected in OCT images. So, the performance of the first grading stage was measured based on the ability of the system to detect these abnormal regions. Included below in Table I is the confusion matrix concerning the detected abnormalities during the first grading stage.

TABLE I
Confusion Matrix for the Detected Abnormalities
in the First Grading state

| | Fluid | Tissue | CH | Merged Layer |
|---|---|---|---|---|
| | Detection | Detection | Detection | Detection |
| TP | 161 | 221 | 230 | 303 |
| TN | 380 | 319 | 310 | 233 |
| FP | 3 | 3 | 1 | 5 |
| FN | 1 | 2 | 4 | 4 |

TP: Total Positive,
TN: Total Negative,
FP: False Positive,
FN: False Negative

As shown in FIG. 7, the training and validation loss during the training stage are measured for the proposed CNN to detect the merged layers. The maximum number of epochs was set to 250 and monitored the value of the validation loss to determine the best training model. Then, the model of the proposed CNN was saved to use for testing unseen data. At that point, the proposed CNN has learned the merged and non-merged layers features.

For the second grading stage, 240° C. T images were used to train and validate the BNN (80 images from normal retina, 80 from non-advanced (early and intermediate) AMD, and 80 from non-AMD disease, (e.g., DR)). The mean square error (MSE) is employed in the BNN to calculate the train and validation loss. The employed BNN includes an early stopping technique that takes validation set errors into account to prevent overfitting. This technique uses the training set to calculate the gradients and update the weights and biases. Also, it monitors the validation errors during the training process and whenever the validation loss exceeds a given number of epochs, the training is halted at this number, then the weights and biases are recorded. In addition, the trained models for eleven OCT layers were saved at the minimum value for MSE. Then, these models were used to test new OCT B-scans and apply the majority voting on the diagnosis for each layer to create the final diagnosis for the OCT image. The best result was found to occur when the majority voting on the output is applied from the last three layers (ellipsoid zone (EZ), outer photoreceptor (OPR) segments, and RPE), where the MSE at these layers are 0.0045, 0.0211, and 0.0004, respectively.

For the third grading stage, 160 images (80 from early AMD and 80 from intermediate AMD) were used. MSE was employed in BNN to obtain the training and validation loss for the eleven OCT layers. The training model is determined when the validation loss provides a minimum value. Then, these models are used to test new subjects, in addition, to applying majority voting on the layers' outputs. Also, the best accuracy to test new subjects was found to occur when the majority voting are applied on the outputs from the last three layers (EZ, OPR, and RPE), which gives the best validation performance at MSE of 0.13, 0.12, and 0.10, respectively.

The disclosed CAD system classifies an OCT image of a retina as indicative of one of seven classes, namely, normal, early AMD, intermediate AMD, GA, inactive wet AMD, active wet AMD, and non-AMD diseases. The CAD system is evaluated based on each class outcome against all other classes. Thus, three evaluation metrics are used for each class: precision, recall, and F1 score. Also, the overall accuracy is calculated for all classes using the known true positive value for each class s. Cohen's Kappa statistic was also used as an evaluation metric as it has the advantage of measuring multiclass problems in addition to imbalanced class problems and depends on the measure of agreement between correct and predicted values. Cohen's Kappa is calculated as follows:

$$Kappa = \frac{T {\sum }_{s = 1}^{H} C {M}_{s s} - {\sum }_{s = 1}^{H} {Cs}_{c orrect} {Cs}_{predict}}{{T}^{2} - {\sum }_{s = 1}^{H} {Cs}_{correct} {Cs}_{predict}} \qquad ( 7 )$$

where CMₛₛ is the diagonal elements in the confusion matrix, H is the total number of classes, T is the total number of subjects in test data, Cs_{correct} is number of elements in the correct class s, and Cs_{predict} is the total number of elements in the predicted class s.

The discussed evaluation metrics were used to calculate the performance of the disclosed CAD system. The performance of the CAD system compared to those of well-known, state of the art deep learning techniques, namely, the pretrained CNNs AlexNet, ResNet101, ResNet50, GoogleNet, and Inception V3. Hyperparameters for these CNNs were tuned and the best hyperparameters selected based on a grid search for the optimizer, whereas the search space was Adam and SGDM. Additionally, the initial learning rate was 0.001 and decreased by a factor of 10 for every 5 iterations. The maximum number of epochs was set to 500 and the validation loss was monitored to get the best-fit training model to use for testing purposes.

As shown in Table II, the overall accuracy of the proposed system gave the highest performance with 90.82% accuracy, while AlexNet, ResNet101, ResNet50, GoogleNet, Swin-Transformer, InceptionV3 and ConvNet achieved accuracies of 70.82, 75.59, 79.26, 74.49, 83.012, 72.11% and 86.48%, respectively. The proposed system achieved the highest kappa score of 89.10%, while the kappa scores were 65.30%, 70.90%, 75.40%, 69.60%, 79.9%, 66.80% and 84.00% % AlexNet, ResNet101, ResNet50, GoogleNet, Swin-Transformer, InceptionV3 and ConvNet, respectively. Differences in classifier performance were found to be statistically significant by Friedman's rank sum test (χ²=29.6, 5 d.f, p=1.7×10⁻⁵). The significance of post hoc comparisons of the proposed classifier with other approaches is shown in the rightmost column of Table II. Bonferroni corrected p-values of the Wilcoxon signed rank statistic are provided.

TABLE II
Comparison Between the Disclosed CAD System and the State of
the Art Deep Learning Techniques

| | | | | | | | | non- | |
|---|---|---|---|---|---|---|---|---|---|
| | | | | | | Inactive | Active | AMD | p- |
| Class | | Normal | Early | Intermediate | GA | wet | wet | disease | value |
| AlexNet | Recall | 81.81% | 59.52% | 80.68% | 69.04% | 61.72% | 65.55% | 71.20% | 1.3 × |
| (Overall | Precision | 94.03% | 55.55% | 78.02% | 61.70% | 61.72% | 67.04% | 70.63% | 10⁻⁵ |
| accuracy = | F1-Score | 87.49% | 57.46% | 79.32% | 65.16% | 61.72% | 66.28% | 70.91% | |
| 70.82%, | | | | | | | | | |
| Kappa = 65.30%) | | | | | | | | | |
| ResNet101 | Recall | 85.71% | 64.28% | 82.95% | 78.57% | 69.13% | 72.22% | 73.60% | 2.0 × |
| (Overall | Precision | 95.65% | 62.79% | 77.66% | 80.48% | 67.47% | 73.86% | 72.44% | 10⁻⁵ |
| accuracy = | F1-Score | 90.40% | 63.52% | 80.21% | 79.51% | 68.28% | 73.03% | 73.01% | |
| 75.59%, | | | | | | | | | |
| Kappa = 70.90%) | | | | | | | | | |
| ResNet50 | Recall | 89.61% | 71.42% | 82.95% | 83.33% | 74.07% | 80.00% | 74.40% | 1.3 × |
| (Overall | Precision | 98.57% | 66.66% | 82.02% | 81.39% | 71.42% | 76.59% | 77.50% | 10⁻⁴ |
| accuracy = | F1-Score | 93.87% | 68.95% | 82.48% | 82.34% | 72.72% | 78.25% | 75.91% | |
| 79.26%, | | | | | | | | | |
| Kappa = 75.40%) | | | | | | | | | |
| GoogleNet | Recall | 84.41% | 66.66% | 85.22% | 73.81% | 66.66% | 70% | 72% | 1.3 × |
| (Overall | Precision | 94.20% | 60.87% | 78.12% | 77.50% | 66.66% | 70.78% | 72.58% | 10⁻⁵ |
| accuracy = | F1-Score | 89.03% | 63.63% | 81.51% | 75.61% | 66.66% | 70.38% | 72.28% | |
| 74.49%, | | | | | | | | | |
| Kappa = 69.60%) | | | | | | | | | |
| Swin- | Recall | 89.47% | 73.68% | 83.33% | 79.54% | 66.66% | 90.24% | 83.33% | 6.7 × |
| Transformer | Precision | 77.27% | 58.33% | 80.00% | 81.39% | 82.35% | 90.73% | 95.57% | 10⁻⁶ |
| (Overall | F1-Score | 82.92% | 65.11% | 81.63% | 80.45% | 73.68% | 90.48% | 88.14% | |
| accuracy = | | | | | | | | | |
| 83.012%, | | | | | | | | | |
| Kappa = 79.9%) | | | | | | | | | |
| InceptionV3 | Recall | 85.71% | 59.52% | 81.81% | 71.42% | 62.96% | 67.77% | 70.4% | 0.0005 |
| (Overall | Precision | 90.41% | 56.81% | 78.26% | 73.17% | 62.96% | 67.59% | 70.96% | |
| accuracy = | F1-Score | 87.99% | 58.13% | 79.99% | 72.28% | 62.96% | 67.67% | 70.67% | |
| 72.11%, | | | | | | | | | |
| Kappa = 66.80%) | | | | | | | | | |
| ConvNet | Recall | 94.11% | 80.00% | 91.66% | 80% | 71.11% | 90.82% | 93.93% | 0.245 |
| (Overall | Precision | 72.72% | 50% | 88% | 93.02% | 94.11% | 96.49% | 90.87% | |
| accuracy = | F1-Score | 81.72% | 61.53% | 89.79% | 86.02% | 81.01% | 90.65% | 94.95% | |
| 86.48%, | | | | | | | | | |
| Kappa = 84.00%) | | | | | | | | | |
| This CAD | Recall | 93.50% | 83.33% | 92.04% | 90.47% | 91.35% | 91.11% | 90.40% | — |
| system | Precision | 91.13% | 80.54% | 94.18% | 86.36% | 88.09% | 91.11% | 95.76% | |
| (Overall | F1-Score | 92.29% | 81.91% | 93.09% | 88.36% | 89.69% | 91.11% | 93.00% | |
| accuracy = | | | | | | | | | |
| 90.82%, | | | | | | | | | |
| Kappa = 89.10%) | | | | | | | | | |

This hybrid system utilizes both deep CNN and ML techniques to diagnose AMD. The main purpose of the proposed system is to differentiate between different AMD grades to help non-retinal specialist physicians to manage these patients and refer them accordingly, if appropriate. However, to generalize our system, it extends capabilities to differentiate between AMD and non-AMD diseases. This has been highlighted using OCTs from DR, MH, ERM, and Stargardt disease. The proposed system is comprehensively composed of three grading stages, unlike other systems which used end-to-end CNN to classify the OCT images. The proposed system uses deep CNN to detect clinically meaningful features related to retina disease. The system uses DeepLabV3+ network to detect fluids (IRF and SRF), subRPE/subretinal tissue, and OH. Also, the system proposes a CNN to detect discontinuities/loss of OCT layers, (merged layers). In addition, the system detects the percentage of fluids and tissue in the macula which is helpful for ophthalmologists in monitoring or treating patients with macular diseases.

Furthermore, ML approaches are utilized through segmenting the undistorted OCT layers and getting discernment relative features. This step requires more investigation in the retinal layers to get discriminate features from the segmented OCT layers. The proposed CAD system has the advantage of detecting the regions of anomaly from the retina region and extracting meaningful features from the OCT layers which are related to macular diseases in the OCT B-scans. Thus, the proposed CAD system can be used as an essential tool by ophthalmologists.

Image data for training and testing the system were acquired by Zeiss Cirrus HD-OCT devices. Longitudinal studies have found that, while there is some variability in quantitative measurements between devices from different manufacturers, the same diagnostic features, e.g. narrowing of a retinal layer, are observed regardless of the device use. In the case of reflectivity-based features, device considerations are more important. Heidelberg Spectralis machines, for example, store reflectivity data on a linear scale, while Zeiss Cirrus pixel values scale logarithmically. There is no documented transformation of one manufacturer's reflectivity scale to another. It is, therefore, expected that the proposed methodology would work with any model of OCT device, provided that the diagnostic component is trained using only data from that scanner.

In other embodiments, optical coherence tomography angiography biomarkers are utilized to diagnose and grade age-related macular degeneration and other eye diseases.

Various aspects of different embodiments of the present disclosure are expressed in paragraphs X1 and X2 as follows:

X1: One embodiment of the present disclosure includes a computer-implemented method for diagnosing age-related macular degeneration (AMD), the method comprising receiving image data including a retina of a subject; generating a diagnosis based on the image data according to a first grading stage which generates a diagnosis of non-AMD disease, active wet AMD, inactive wet AMD, geographic atrophy, or no diagnosis; generating a diagnosis based on the image data according to a second grading stage which, if the diagnosis of the first grading stage is no diagnosis, generates a diagnosis of non-AMD disease, normal, or one of early AMD or intermediate AMD; and generating a diagnosis based on the image data according to a third grading stage which, if the diagnosis of the second grading stage is one of early AMD or intermediate AMD, generates a diagnosis or early AMD or intermediate AMD.

X2: Another embodiment of the present disclosure includes A computer-implemented method for classifying a retina, the method comprising processing image data including a subject retina to extract retinal abnormalities; classifying the subject retina based on the extracted retinal abnormalities according to a first grading stage; segmenting the subject retina into a plurality of retinal layers; extracting at least one feature from each of the segmented layers; classifying the subject retina based on the extracted at least one feature according to a second grading stage; generating at least one second order feature based on the extracted at least one feature; and classifying the subject retina based on the extracted at least one feature and the at least one second order feature according to a third grading stage.

Yet other embodiments include the features described in any of the previous paragraphs X1 or X2 combined with one or more of the following aspects:

Extracting retinal abnormalities from the image data and generating, using a rule-based classifier, the diagnosis of the first grading stage based on the extracted retinal abnormalities.

Wherein the retinal abnormalities are one or more of subretinal tissue, sub-retinal pigment epithelium tissue, intraretinal fluid, subretinal fluid, choroidal hypertransmission, and merged retinal layers.

Processing the image data to segment the retina into a plurality of retinal layers; extracting at least one feature from the segmented retina; and generating, using a machine learning classifier, the diagnosis of the second grading stage based on the extracted at least one feature.

Wherein the at least one feature is at least one of retinal curvature, thickness and reflectivity.

Wherein the at least one feature is extracted from each of the plurality of retinal layers.

Generating a diagnosis for each of the plurality of retinal layers based on the extracted at least one feature.

Wherein the diagnosis of the second grading stage is generated by majority voting of the diagnoses generated for the plurality of retinal layers.

Wherein said processing the image data to segment the retina into the plurality of retinal layers occurs after said generating the diagnosis based on the image data according to the first grading stage.

Generating, using a machine learning classifier, the diagnosis of the third grading stage based on the extracted at least one feature and at least one second order feature generated based on the extracted at least one feature.

Wherein the at least one feature includes reflectivity and optionally includes retinal curvature and thickness, and wherein the at least one second order feature is generated based on the reflectivity.

Wherein the image data is includes optical coherence tomography (OCT) image data.

Wherein the classification according to the first grading state is one of non-AMD disease, active wet AMD, inactive wet AMD, geographic atrophy, or no classification.

Wherein, if the classification according to the first grading stage was no classification, the classification according to the second grading stage is one of non-AMD disease, normal, or one of early AMD or intermediate AMD.

Wherein, if the classification according to the second grading stage was one of early AMD or intermediate AMD, the classification according to the third grading stage is either early AMD or intermediate AMD.

Wherein the retinal abnormalities are one or more of subretinal tissue, sub-retinal pigment epithelium tissue, intraretinal fluid, subretinal fluid, choroidal hypertransmission, and merged retinal layers.

Wherein the at least one feature is at least one of retinal curvature, thickness and reflectivity.

Wherein the at least one feature includes reflectivity and optionally includes retinal curvature and thickness, and wherein the at least one second order feature is generated based on the reflectivity.

Wherein the classification according to the first grading stage is performed using a rules-based classifier and wherein the classification according to the second and third grading stages are performed using machine learning classifiers.

The foregoing detailed description is given primarily for clearness of understanding and no unnecessary limitations are to be understood therefrom for modifications can be made by those skilled in the art upon reading this disclosure and may be made without departing from the spirit of the invention.

1) A computer-implemented method for diagnosing age-related macular degeneration (AMD), the method comprising:
receiving image data including a retina of a subject;
generating a diagnosis based on the image data according to a first grading stage which generates a diagnosis of non-AMD disease, active wet AMD, inactive wet AMD, geographic atrophy, or no diagnosis;
generating a diagnosis based on the image data according to a second grading stage which, if the diagnosis of the first grading stage is no diagnosis, generates a diagnosis of non-AMD disease, normal, or one of early AMD or intermediate AMD; and
generating a diagnosis based on the image data according to a third grading stage which, if the diagnosis of the second grading stage is one of early AMD or intermediate AMD, generates a diagnosis or early AMD or intermediate AMD.

2) The method of claim 1, further comprising
extracting retinal abnormalities from the image data; and
generating, using a rule-based classifier, the diagnosis of the first grading stage based on the extracted retinal abnormalities.

3) The method of claim 2, wherein the retinal abnormalities are one or more of subretinal tissue, sub-retinal pigment epithelium tissue, intraretinal fluid, subretinal fluid, choroidal hypertransmission, and merged retinal layers.

4) The method of claim 1, further comprising
processing the image data to segment the retina into a plurality of retinal layers;
extracting at least one feature from the segmented retina; and
generating, using a machine learning classifier, the diagnosis of the second grading stage based on the extracted at least one feature.

5) The method of claim 4, wherein the at least one feature is at least one of retinal curvature, thickness and reflectivity.

6) The method of claim 4, wherein the at least one feature is extracted from each of the plurality of retinal layers.

7) The method of claim 4, further comprising
generating a diagnosis for each of the plurality of retinal layers based on the extracted at least one feature and
wherein the diagnosis of the second grading stage is generated by majority voting of the diagnoses generated for the plurality of retinal layers.

8) The method of claim 4, wherein said processing the image data to segment the retina into the plurality of retinal layers occurs after said generating the diagnosis based on the image data according to the first grading stage.

9) The method of claim 4, further comprising generating, using a machine learning classifier, the diagnosis of the third grading stage based on the extracted at least one feature and at least one second order feature generated based on the extracted at least one feature.

10) The method of claim 4, wherein the at least one feature includes reflectivity and optionally includes retinal curvature and thickness, and wherein the at least one second order feature is generated based on the reflectivity.

11) The method of claim 1, wherein the image data is includes optical coherence tomography (OCT) image data.

12) A computer-implemented method for classifying a retina, the method comprising:
processing image data including a subject retina to extract retinal abnormalities;
classifying the subject retina based on the extracted retinal abnormalities according to a first grading stage;
segmenting the subject retina into a plurality of retinal layers;
extracting at least one feature from each of the segmented layers;
classifying the subject retina based on the extracted at least one feature according to a second grading stage;
generating at least one second order feature based on the extracted at least one feature; and
classifying the subject retina based on the extracted at least one feature and the at least one second order feature according to a third grading stage.

13) The method of claim 12, wherein the classification according to the first grading state is one of non-AMD disease, active wet AMD, inactive wet AMD, geographic atrophy, or no classification.

14) The method of claim 13, wherein, if the classification according to the first grading stage was no classification, the classification according to the second grading stage is one of non-AMD disease, normal, or one of early AMD or intermediate AMD.

15) The method of claim 14, wherein, if the classification according to the second grading stage was one of early AMD or intermediate AMD, the classification according to the third grading stage is either early AMD or intermediate AMD.

16) The method of claim 12, wherein the retinal abnormalities are one or more of subretinal tissue, sub-retinal pigment epithelium tissue, intraretinal fluid, subretinal fluid, choroidal hypertransmission, and merged retinal layers.

17) The method of claim 12, wherein the at least one feature is at least one of retinal curvature, thickness and reflectivity.

18) The method of claim 12, wherein the at least one feature includes reflectivity and optionally includes retinal curvature and thickness, and wherein the at least one second order feature is generated based on the reflectivity.

19) The method of claim 12, wherein the classification according to the first grading stage is performed using a rules-based classifier and wherein the classification according to the second and third grading stages are performed using machine learning classifiers.
